(19) United States
(12) Patent Application Publication
(10) Pub. No.: US 2023/0405285 A1
(43) Pub. Date: Dec. 21, 2023

(54) Guide Sheath for Vascular Procedures

(71) Applicant: TZ MEDICAL, INC., Portland, OR (US)

(72) Inventor: Austin Ziegler, Tigard, OR (US)

(73) Assignee: TZ MEDICAL, INC., Portland, OR (US)

(21) Appl. No.: 17/843,829

(22) Filed: Jun. 17, 2022

(51) Int. Cl. A61M 25/06

(52) U.S. Cl. CPC A61M 25/0662

(57) ABSTRACT

A guide sheath for vascular procedures has a flat seal that allows insertion and removal of catheters or the like, sealing the vascular system against the exterior. In use, the flat surface can be easily wiped to remove any dried or clotted blood prior to insertion of the catheter.

### BACKGROUND

This disclosure relates vascular system procedures, more particularly to a sheath used in an endovascular procedure.

In certain medical procedures, therapeutic and diagnostic catheters as well as ancillary devices are introduced into a patient's endovascular system through a sheath. Initial access is gained percutaneously through insertion of an introducer needle into the vein or artery. A small wire is then placed through the needle and advanced into the artery or vein to secure access. The needle is then removed and a dilator, inside a sheath, is inserted over the wire and advanced through the skin and into vein or artery. The wire and dilator are then removed leaving the sheath body as a sealed hemostatic conduit to facilitate the insertion of catheters and ancillary devices into the vascular system.

After the procedure is completed, the sheath is removed and hemostasis of the access site is achieved by applying manual pressure or the facilitation of a closure device.

FIGS. 1-3 illustrate partial perspective views of a sheath proximal hub and gasket 112 that is inserted in the vein or artery at the tapered end on the right of the drawing. An opening in the hub/gasket well 114 in the sheath allows access of a catheter 116 or ancillary device to be inserted into the body of the sheath.

Once the catheter is removed, blood can pool at the opening 114 and coagulate. When a catheter is later reintroduced into the opening, there is a risk of clot formation from the opening 114 being pushed into the vascular system potentially causing risk or injury to the patient. A side port flush valve 118 is provided so the any clot located in the shaft area between the sheath and the catheter can be removed before reinserting a catheter. While effective, in removing a majority of clot there remains the potential for clot formation in the indented gasket well of the hub 114 requiring continuous observation by the operator for clot formation and introduction into the vascular system during catheter and ancillary device exchanges.

### SUMMARY

In accordance with the disclosure, a vascular sheath is provided that prevents clot formation at gasket well lowering patient complication risk.

The subject matter of the present technology is particularly pointed out and distinctly claimed in the concluding portion of the specification. However, both the organization and method of operation, together with further advantages and embodiments thereof, may best be understood by reference to the following description taken in connection with accompanying drawings wherein like reference characters refer to like elements.

### BRIEF DESCRIPTION OF THE DRAWINGS

FIG. 1 is a partial perspective view of a catheter introducer device in accordance with the prior art;

FIG. 2 is a perspective view of a guide sheath in accordance with the prior art, as a catheter is being inserted or withdrawn;

FIG. 3 is a view of cutout view of the catheter introducer mated to the hub of the device in accordance with the prior art of guide sheath in FIG. 2;

FIG. 4 is a perspective view of the guide sheath in accordance with the present disclosure;

FIG. 5 is a detail view of a portion of the guide sheath identified in FIG. 4;

FIG. 6 is an exploded a view of the guide sheath of FIG. 4;

FIG. 7 is a detail view of a portion of the guide sheath in FIG. 6;

FIG. 8 is a view of the guide sheath of FIG. 42 looking in the direction of arrow 6 of FIG. 4;

FIG. 9 is a sectional view of the guide sheath taken along line 9-9 of FIG. 5 and line 7 of FIG. 8;

FIG. 10 is a detail view of the guide sheath of FIG. 9;

FIG. 11 is a perspective view of the sheath from the gasket face side with a trocar/inserter being inserted or withdrawn;

FIG. 12 is a perspective view of the sheath from behind the gasket face side with a trocar/inserter being inserted or withdrawn;

FIG. 13 is a perspective view from the view of FIG. 11 with the trocar/inserter fully inserted and secured to the guide sheath;

FIG. 14 is a perspective view of the device with flushing line attached to the flushing port;

FIG. 15 is a perspective view of the device in FIG. 14, with a trocar/inserter partially inserted into the sheath; and

FIG. 16 is a perspective view of the device in FIG. 14, with a trocar/inserter fully inserted into the sheath.

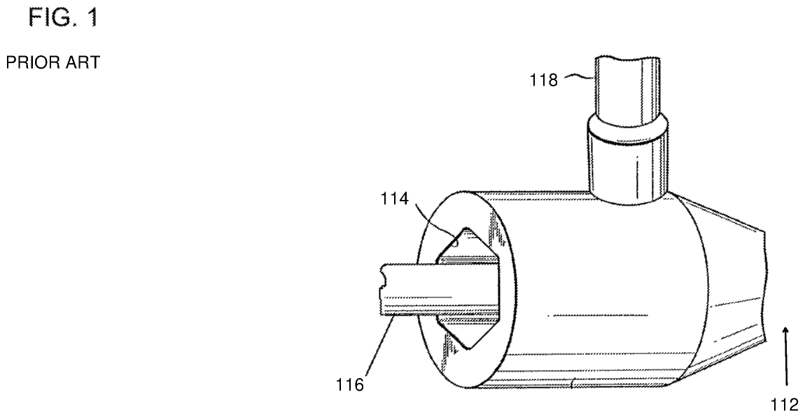

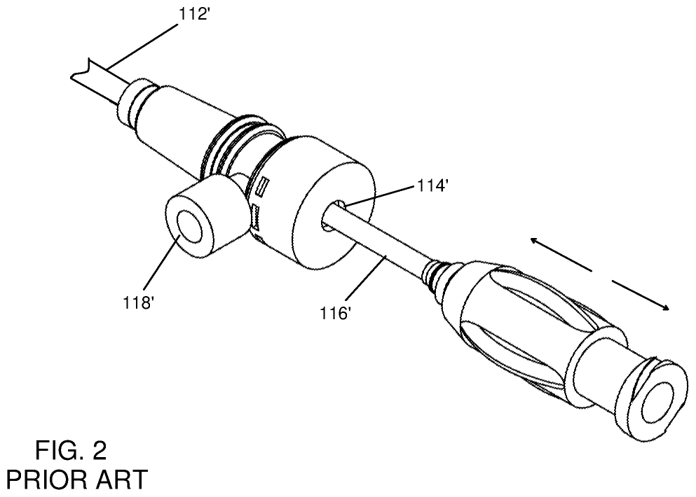

### DETAILED DESCRIPTION

The system according to a preferred embodiment of the present disclosure comprises a guide sheath adapted to prevent clot formation in the gasket well of the sheath hub.

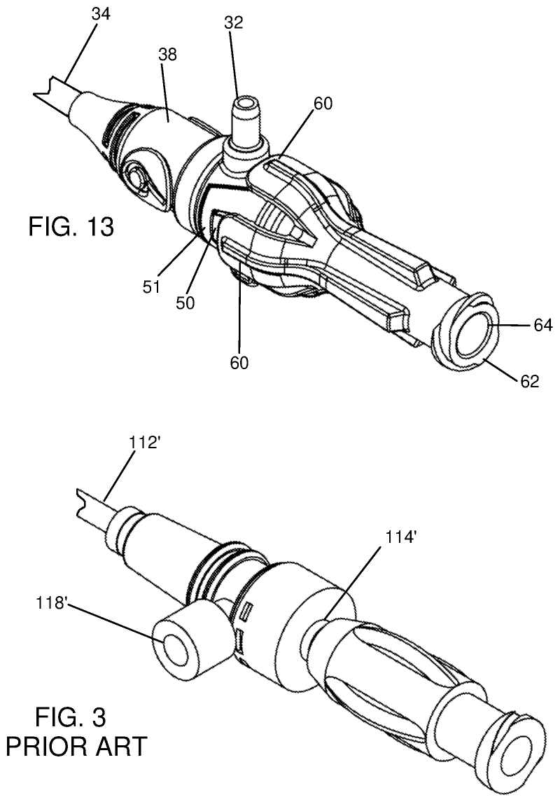

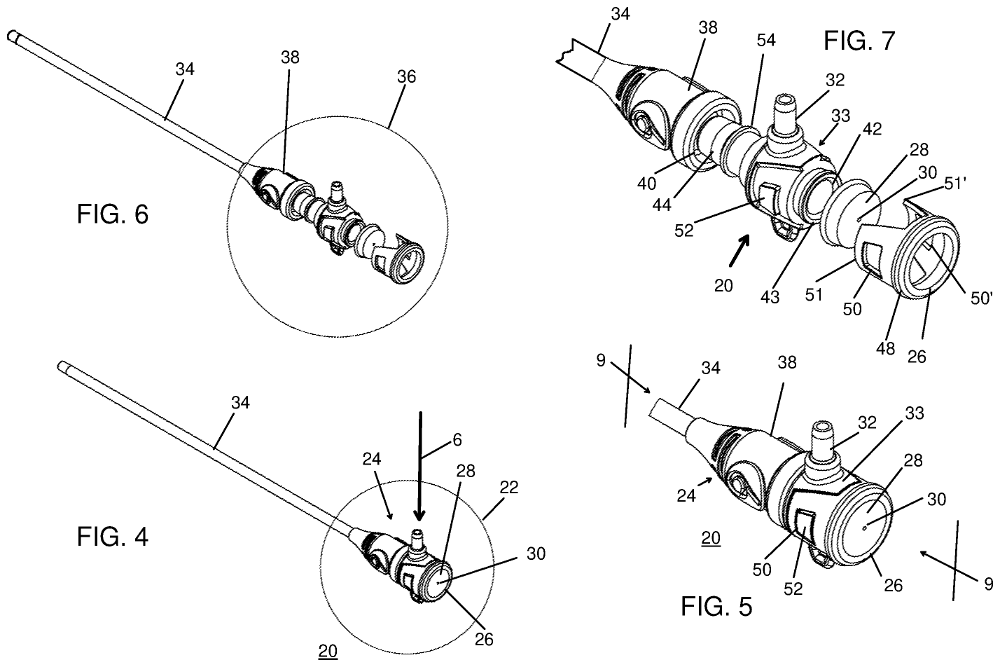

Referring to FIG. 4, a perspective view of a guide sheath 20 in accordance with the disclosure, and FIG. 5, a magnified view of the area 22 of the guide sheath of FIG. 4, the sheath comprises a body (hub) 24, with opening port 26 adapted to receive a catheter therein for access to the patient's vascular system. At the face of the opening 26, a gasket 28 covers the face of the opening port 26, providing a smooth and substantially flat exterior surface, while allowing the catheter to be introduced through the gasket such as by puncturing the gasket. A central marker 30, such as a visible dot or slight concavity in the gasket can be provided to help guide insertion of a catheter near the center of the gasket. The gasket is such that on removal of the catheter, the punctured opening seals so that little or no leakage occurs on removal of the catheter (or on insertion), much in the manner that gaskets on medicine vials allow entry and removal of a needle. Since the gasket 28 lies flat on the surface of the hub and is held by the snap cover FIG. 7 48, there is no well or indentation to allow clot to form. Further, prior to insertion of a vascular device, the exterior surface of the gasket can be wiped, such as with a sterile wipe, to remove any blood that may have collected on the surface thus reducing the possibility of coagulation and decreasing the chance of a clot entering the vascular system with the insertion of the vascular device.

In use, the hollow shaft 34 of the guide sheath is inserted into the patient, such as via a vein or artery, and thus the device allows easy introduction or removal of a catheter into the patient by way of the opening 26 and gasket 28.

A traditional type side port flush or injection port 32 can also be provided to allow flushing or introduction of medicine as in the prior art, should it become necessary or be necessary to comply with procedural requirements.

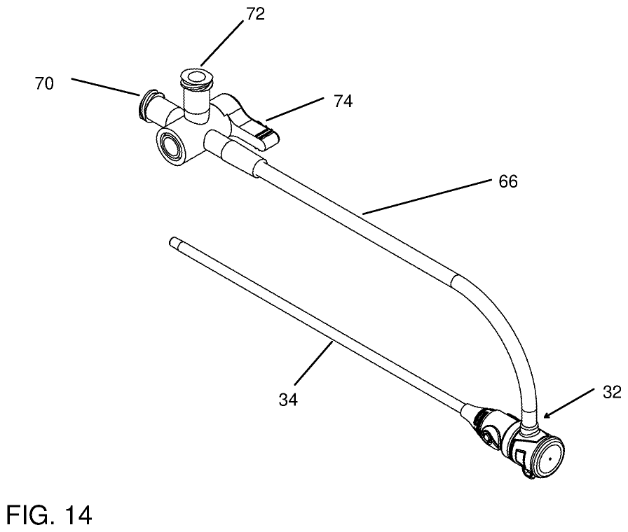

Referring to FIGS. 6 and 7, where FIG. 6 is an exploded view of the guide sheath of FIG. 4 and FIG. 7 is a detailed view of the area 36 of the guide sheath in FIG. 6, the body (hub) 24 is constructed of several sub-components, the distal sheath body 38, which is formed and is concentric with hollow shaft 34, having a central opening 40. The side port flush or injection port 32 is formed as a 2 nd modular piece 33 with a central through port 42 and a distal extension 44 that snap fits into the opening 40, for providing fluid communication through a pig tail tube with stopcock (shown with reference to FIGS. 14-16) connected to 32 the guide sheath to the hollow shaft tip portion. A gasket mounting flange 43 at the proximal end of portion 33 receives the gasket 28 for securing the gasket to the portion 33. Opening 26 is formed on a snap cover 48, the snap cover having left and right engagement openings 50, 50′ formed on legs 51, 51′ that extend from the opening 26 that engage with corresponding raised portions 52 on the left side of portion 33 (and counterpart raised portion 52′ on the right side), so that the snap cover 48 securely engages with the body 33. By pressing the various components together, an assembled guide sheath is provided.

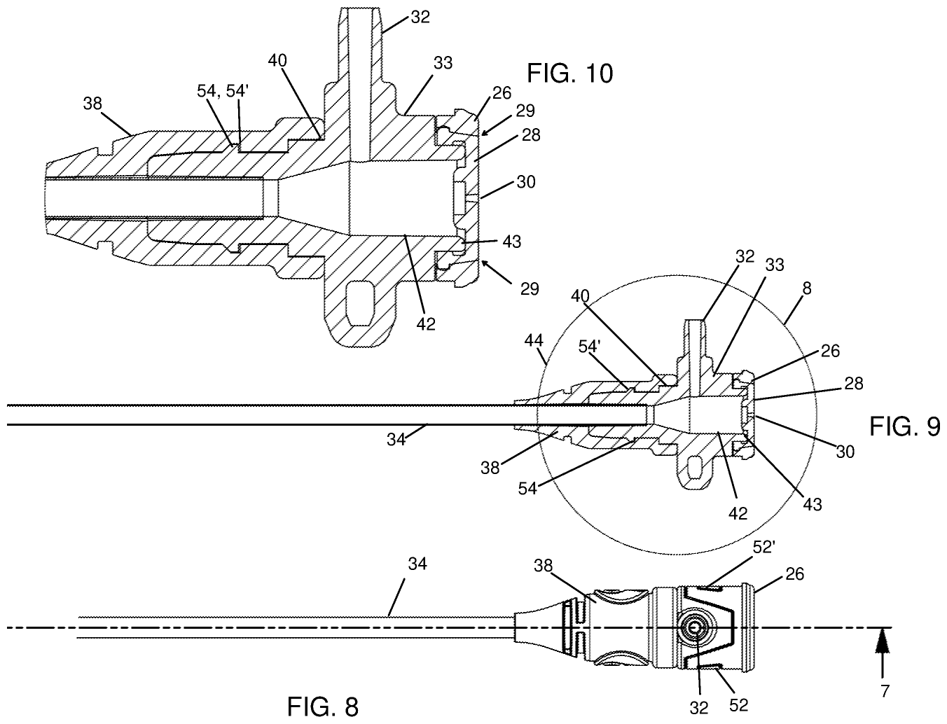

Referring to FIGS. 8-10, FIG. 8 being a view of the guide sheath of FIG. 4 looking in the direction of arrow 6 of FIG. 4, FIG. 9 being a cross-sectional view of the guide sheath taken along line 9-9 of FIG. 5 and FIG. 10 being a detailed view of the area in circle 44 of FIG. 9, the engagement of the various components of the guide sheath can be observed. Portion 33 has a raised circumferential ring portion 54 that allows snap together assembly by interaction with corresponding ring slot 54′ in the interior of body 38, securing portion 33 and body 38 together when the guide sheath is assembled. Port flush (tubing with stop-cock shown in FIGS. 14-16) or injection port 32 has a central conduit allowing fluid communication with the interior 42 of the guide sheath so that flushing or introduction of medicine can be accomplished through the valve/port. Gasket 28 fits on top of circumferential mounting flanges 43 and opening cover 26 fits over the top thereof, engaging with the gasket to hold the gasket to the body 33, while providing a smooth surface transition at 29 between the gasket and the opening cover.

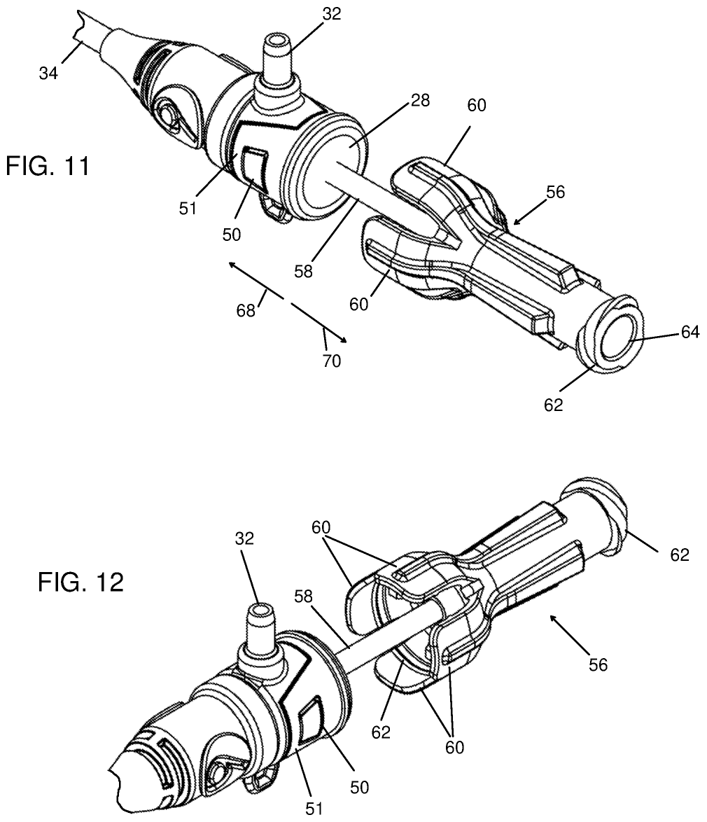

Referring to FIG. 11 and FIG. 12, perspective views of the sheath from the gasket face side and from behind the gasket face side, with a catheter being inserted or withdrawn and FIG. 13 is a perspective view from the view of FIG. 11 with the catheter fully inserted and secured to the guide sheath. The introducer/trocar 56 has a dialator portion 58, which has been inserted through the gasket 28 so as to allow the dialator to extend into the portion 34 of the guide sheath. The introducer/trocar 56 includes flange portions 60 (four such flange portions in the illustrated embodiment) that define an open region 62 of size and shape to fit over the body of the guide sheath so as to secure the engagement of the introducer/trocar to the guide sheath with a frictional fit, for example, as in FIG. 13, whereby the introducer/trocar is grasping the body of the guide sheath. The introducer/trocar includes a threaded distal end portion 62 for attachment of other devices thereto. Central opening 64 of the introducer/trocar allows insertion of other devices therein.

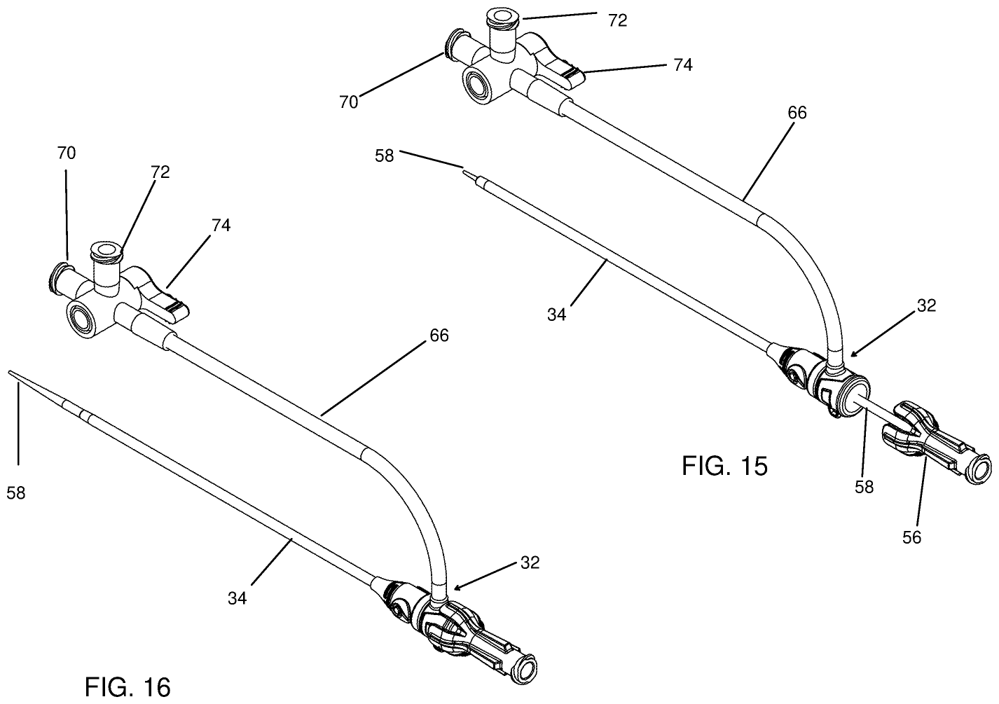

Referring to FIGS. 14-16, showing the device with flush tube as used in procedures, FIG. 14 showing the device with flushing line attached to the flushing port, and FIGS. 15 and 16 showing the device and flushing tube with a trocar/inserter partially and fully inserted into the sheath. In use a pig tail flush tube 66 is connected at one end to side port flush or injection port 32. The opposite end of the flush tube 66 carries a two port flush valve 68, having ports 70, 72. Control 74 is rotatable for connecting either of port 70 or 72 to the flush tube (or for closing both off to the flush tube). For primary use of the flush port, one of the ports 70, 72 will be capped off and not used during the procedure, while the other is connected to a syringe, for example, containing a flushing solution, such as saline. The control 74 is rotated to connect the capped port to the flush tube, for closing off the flush tube. For flushing or clot removal, the control is rotated to connect the syringe port to the flush tube, allowing removing of blood and clots as well as allowing flushing with a saline type of solution.

Some of the advantages to the design herein are that the face of the device where the catheter is inserted is flat and provides a smooth surface that can be easily wiped clean and does not have a pocket or well where blood can pool (and coagulate). Further, the manner in which introducer/trocar joins to the sheath is novel as the introducer/trocar clasps over the entire structure rather than connecting to the hole/well in the traditional design.

Therefore, the guide sheath in accordance with the disclosure provides reduced risk of introducing a clot to the patient's vascular system, and provides the possibility of simplified procedures, not requiring flushing of the guide sheath with each re-introduction of a catheter.

While a preferred embodiment of the technology has been shown and described, it will be apparent to those skilled in the art that many changes and modifications may be made without departing from the broader aspects. The appended claims are therefore intended to cover all such changes and modifications as fall within the true spirit and scope of the technology.

What is claimed is:

1. A guide sheath for vascular procedures, comprising:
a body having an interior passage therethrough, including receiving orifice for insertion of an introducer/trocar therein;
a sealing gasket for sealing said interior passage to an external atmosphere at said receiving orifice; and
a securement member for securing said gasket to said body, wherein the engagement of said securement member and said sealing gasket provides a substantially flat junction between the sealing member and gasket at a face external to the interior passage.

2. The guide sheath according to claim 1, wherein said introducer/trocar comprises an insertion dialator for penetrating said gasket and extending into said interior passage; and
plural engagement flanges adapted for securing said introducer/trocar to said guide sheath body.

3. The guide sheath according to claim 2, wherein said securement member and guide sheath body present a circular profile and said engagement flanges present a corresponding circular opening to fit over the circular profile when said introducer/trocar and guide sheath body are engaged.

4. The guide sheath according to claim 1, further comprising a flushing port in communication with said interior passage for allowing flushing or clot removal.

5. The guide sheath according to claim 4, further comprising a flush tube in communication with said flushing port.

6. The guide sheath according to claim 5, further comprising a valve in communication with said flush tube for alternatively closing said flush tube or connecting said flush tube to a flushing device.

7. The guide sheath according to claim 1, further comprising a gasket mounting flange on said body for mounting said gasket thereto.

8. The guide sheath according to claim 7, further comprising a cover member for securing said gasket to said body.

9. A access port for vascular procedures, comprising:
a body having an opening to a passage, a first end of the passage for insertion to a vascular system, a second end of the passage for receiving a catheter therein for passing through said passage;
a seal for covering said opening at the second end of said passage, wherein said seal provides a substantially flat face external to the guide sheath for inhibition of collection or pooling of blood at said opening;
an introducer/trocar for insertion to said opening through said seal; and
a frictional engagement member for securing said introducer/trocar in engagement with said body.

10. The access port according to claim 9, wherein said frictional engagement member comprises plural extending members on said introducer/trocar defining an open space into which said body frictionally engages for securing.

11. The access port according to claim 9, further comprising a securement member for securing said seal to said body, wherein the engagement of said securement member and said seal provides a substantially flat junction between the sealing member and gasket at a face external to the passage.

12. The access port according to claim 9, further comprising a flushing port in communication with said passage for allowing flushing or clot removal.

13. The access port according to claim 12, further comprising a flush tube in communication with said flushing port.

14. The access port according to claim 13, further comprising a valve in communication with said flush tube for alternatively closing said flush tube or connecting said flush tube to a flushing device.

15. A method of providing a guide sheath for vascular procedures, comprising:
providing a receiving orifice in a guide sheath body for insertion of a catheter therein; and
providing a sealing membrane covering said receiving orifice for sealing an interior portion of the guide sheath body from the atmosphere while allowing insertion and removal of the catheter, said sealing membrane presenting a substantially flat face external to the guide sheath body.

16. The method according to claim 15, further comprising providing plural engagement flanges adapted for securing said catheter to said guide sheath body.

17. The method according to claim 15, further comprising providing a flushing port in communication with an interior of said receiving orifice allowing flushing or clot removal.

18. The method according to claim 15, further comprising providing a flush tube in communication with said flushing port.

19. The method according to claim 18, further comprising providing a valve in communication with said flush tube for alternatively closing said flush tube or connecting said flush tube to a flushing device.
